(19) United States
(12) Patent Application Publication
(10) Pub. No.: US 2025/0135103 A1
(43) Pub. Date: May 1, 2025

(54) Roller Assembly and Enteral Feeding Pump

(71) Applicant: Medcaptain Medical Technology Co., Ltd., Shenzhen (CN)

(72) Inventors: Pan HU, Shenzhen (CN); Zhiwu DENG, Shenzhen (CN)

(73) Assignee: Medcaptain Medical Technology Co., Ltd., Shenzhen (CN)

(21) Appl. No.: 18/894,036

(22) Filed: Sep. 24, 2024

(30) Foreign Application Priority Data
Oct. 27, 2023 (CN) CN202311409266.7

(51) Int. Cl. A61M 5/142; A61J 15/00

(52) U.S. Cl. CPC A61M 5/14232; A61J 15/0003

(57) ABSTRACT

An enteral feeding pump includes a pump main body, a roller assembly. The roller assembly includes a housing and a locking structure connected to the housing. The locking structure includes a switch member and a cooperating locking member. The roller assembly is detachably connected to the pump main body, and the locking member is configured to be in an unlocked position or a locked position depending on when the switch member is triggered. The roller assembly is thereby easy to assemble and disassemble for cleaning and maintenance.

### REFERENCE TO RELATED APPLICATIONS

The invention claims priority of Chinese Patent Application No. 202311409266.7, entitled “Roller Assembly and Enteral Feeding Pump” filed with the China National Intellectual Property Administration on Oct. 27, 2023, which is incorporated herein by reference in its entirety.

### TECHNICAL FIELD

This invention generally relates to medical devices, and more particularly, to apparatuses for enteral feeding.

### BACKGROUND ART

An enteral feeding apparatus is a nutrition infusion pump provided for nasal feeding, which can input and provide substances such as water, a nutritional fluid and homemade meal milk of a certain concentration to a patient through a nasal feeding tube, thereby improving the nutritional status of a patient.

The enteral feeding apparatus generally includes an enteral feeding pump and a roller mounted on the enteral feeding pump. An infusion tube is then wound at least partially around the roller, which is driven to rotate by a drive device in the enteral feeding pump so as to squeeze the tube and promote the flow of liquid in the infusion tube. As an important functional component of the enteral feeding pump, the roller needs to be frequently disassembled from the main body of the enteral feeding pump during long-term use to clean or repair and replace the roller, after which the roller is reinstalled on the enteral feeding pump.

However, traditional rollers are connected to the main body of the enteral feeding pump through screws and other fasteners, which are not easy to install and remove, and cause difficulties in cleaning and maintenance.

### SUMMARY OF THE INVENTION

In order to solve at least one problem mentioned in the background art, the invention provides a roller assembly and an enteral feeding pump, where the roller assembly is easy to assemble and disassemble for cleaning and maintenance.

One aspect of the invention provides a roller assembly comprising a housing, and a locking structure connected to the housing; wherein the locking structure comprises a switch member and a cooperating locking member. The locking member is configured to be in an unlocked state or a locked state when the switch member is triggered.

In one possible embodiment, a positioning groove is provided on a side of the housing, and the locking member is configured to extend into the positioning groove when in the locked state, and to move in a direction away from a centerline of the positioning groove when in the unlocked state.

In one possible embodiment, the housing is provided with an accommodating cavity, and the locking structure is mounted in the accommodating cavity. The housing is further provided with a first opening and a second opening, which are both connected to the accommodating cavity, A switch member is movably disposed within the first opening and the second opening is connected to the positioning groove. The locking member is movably disposed within the second opening.

In one possible embodiment, the switch member comprises a pressing portion and a driving portion, which are connected to each other. The pressing portion is movably disposed within the first opening. The driving portion abuts against the locking member to drive the locking member to move toward a side back away from the second opening.

In one possible embodiment, the locking member is provided with a guide groove and the driving portion is movably connected within the guide groove. A groove wall of the guide groove is provided with a first guide portion that is arranged at an inclined angle, and the driving portion is provided with a second guide portion that is arranged at an inclined angle. The first guide portion cooperates with the second guide portion to drive the locking member to move toward the side back away from the second opening.

In a possible embodiment, the locking structure further comprises a first elastic member, two ends of which resist the switch member and the inner wall of the accommodating cavity, the first elastic member being configured to drive the switch member toward the side of the first opening.

In one possible embodiment, the locking structure further comprises a first elastic member. Opposite ends of the first elastic member abut against the switch member and an inner wall of the accommodating cavity, respectively, and the first elastic member is configured to drive the switch member to move toward the first opening.

In one possible embodiment, the housing comprises a roller body and an end cap which are detachably connected to each other. The accommodating cavity is disposed between the roller body and the end cap and the first opening is provided in the end cap. The positioning groove is provided on a side of the roller body back away from the end cap.

In one possible embodiment, a side wall of the roller body is provided with a mounting port connected to the accommodating cavity, and the mounting port is provided with a detachable stopper.

In one possible embodiment, the roller assembly further comprises a plurality of rollers, wherein the roller body comprises a main body portion and a fix portion which are connected to each other, the plurality of rollers are disposed between the fix portion and the end cap and spaced circumferentially around the main body portion, and the rollers are rotatable about their own rotational axis.

In one possible embodiment, the end cap is provided with a snap fastener, and the roller body is provided with a first snap-fit groove adapted to the snap fastener.

In one possible embodiment, an avoidance surface is provided at an end of the locking member extending into the positioning groove.

Another aspect of the invention provides an enteral feeding pump comprising a pump main body and a roller assembly as described hereinbefore, wherein the roller assembly is detachably connected to the pump main body.

In one possible embodiment, the pump main body is provided with a pump main shaft, and a locking member of the roller assembly is connected to the pump main shaft in the locked state.

In one possible embodiment, the pump main shaft is provided with a second snap-fit groove, and the locking member of the roller assembly extends into the snap-fit groove when in the locked state.

In one possible embodiment, the roller assembly is provided with a positioning groove, which is inserted into and mates with the pump main shaft. An inner side wall of the positioning groove is provided with a first locating surface, an outer side wall of the pump main shaft is provided with a second locating surface, and the first locating surface mates with the second locating surface, so that the pump main shaft drives the roller assembly to rotate.

In one possible embodiment, the pump main body is further provided with a detecting member, which is configured to detect a state of the roller assembly and send a signal to a control unit of the pump main body in case of abnormality of the roller assembly.

The invention provides a roller assembly and an enteral feeding pump, where the roller assembly is detachably mounted on the pump main body of the enteral feeding pump. The roller assembly is provided with a locking structure connected to its housing, and the locking structure is provided with a switch member and a locking member, which cooperate with each other. The switch member can be triggered by an operator. When the switch member is not triggered, the locking member is in the locked state, and the roller assembly is locked on the pump main body. When the switch member is triggered, the locking member is in the unlocked state, the roller assembly is disconnected from the pump main body, and the roller assembly can be removed from the pump main body. In this way, the connection between the roller assembly and the pump main body is more flexible, and installation and removal of the roller assembly on the pump main body do not require the use of tools. The installation and removal of the roller assembly is more efficient, and it is easy to clean and maintain the roller assembly.

### BRIEF DESCRIPTION OF THE DRAWINGS

In order to more clearly illustrate the technical solutions in the embodiments the accompanying drawings to be used in the description of the embodiments or prior art will be briefly introduced below, and it will be obvious that the accompanying drawings in the following description are some embodiments of the invention. For a person of ordinary skill in the art, other accompanying drawings can be obtained from these accompanying drawings without creative labor.

FIG. 1 shows a schematic structural diagram of an enteral feeding pump provided by an embodiment of the invention.

FIG. 2 shows a structural view of the enteral feeding pump of FIG. 1 after removal of an infusion assembly.

FIG. 3 shows a schematic structural diagram of the roller assembly in one view provided by embodiments of the invention.

FIG. 4 shows another view of a schematic structural diagram of the roller assembly of FIG. 3.

FIG. 5 shows an exploded view of the roller assembly of FIG. 3.

FIG. 6 shows a cross-sectional view of the roller assembly along line B-B of FIG. 3 when the locking member is in a locked state.

FIG. 7 shows a cross-sectional view of the roller assembly of FIG. 3 when the locking member is in an unlocked state.

FIGS. 8a-8c together show a structural diagram of the switch member and the cooperating locking member provided by an embodiment of the invention.

FIG. 9 shows a cross-sectional view of the roller assembly of along line A-A FIG. 3 when the locking member is in the locked state.

### DETAILED DESCRIPTION

### Description of the Reference Numbers

1—enteral feeding pump;
100—pump main body; 200—roller assembly; 300—infusion assembly; 400—pump door;
110—support pad; 120—display screen; 130—button; 140—indicator light; 150—pump main shaft; 210—housing; 220—switch module; 230—locking module;
151—second snap-fit portion; 151a—first snap-fit groove; 152—second locating surface; 211—roller main body; 212—end cap; 213—roller; 221—switch member; 222—first elastic member; 231—locking member; 232—second elastic member;
1511—second limiting surface; 2101—positioning groove; 2101a—positioning portion; 2111—body portion; 2112—fix portion; 2121—first opening; 2122—snap fastener; 2131—rotor shaft; 2211—pressing portion; 2212—driving portion; 2311—guide groove; 2312—first snap-fit portion; 2312a—snap-fit tab;
21011—second opening; 21012—locating tab; 21013—first locating surface; 21111—mounting port; 21112—stopper; 21113—second snap-fit groove; 22121—second guide portion; 22122—abutment portion; 23111—first guide portion; 23112—vertical portion; 23121—first limiting surface; 23122—avoidance surface; 21111a—guide groove.

Improving the nutritional status of patients is at the core of much disease treatment. Clinical nutrition is divided into enteral nutrition and parenteral nutrition. Enteral nutrition is more conducive to the patient's metabolism and the utilization of nutrients in the human body; it can also safely and effectively promote and maintain the intact structure and function of the gastrointestinal tract, protect the gastric mucous barrier, reduce the number of complications, and lower the cost of treatment.

In the clinical setting, water, nutrient solutions and a certain concentration of other substances such as homemade rice milk are generally delivered to the patient using an enteral feeding pump, which is also known as a nutrition infusion pump, in order to provide patients with nutrition. As an important functional component of the enteral feeding pump, a roller needs to be frequently disassembled, removed from the main body of the enteral feeding pump, and cleaned, or it needs to be repaired and replaced. The cleaned (or repaired and re-replaced) roller then needs to be re-installed on the main body of the enteral feeding pump.

In a traditional enteral feeding pump, the roller is fixedly connected to the main body of the enteral feeding pump by screws, bolts and other fasteners, and it is necessary to use a tool, and sometimes even a special tool, to install the roller on the main body of the enteral feeding pump, or to remove the roller from the main body of the enteral feeding pump. This obviously complicates the installation and removal of the roller, which may, moreover, cause inconvenience to the user and the maintenance personnel. Without the proper tools, it is even impossible to implement cleaning or maintenance of the roller.

In view of this, embodiments of the invention provide a roller assembly and an enteral feeding pump, where the roller assembly is detachably mounted on the pump main body of the enteral feeding pump and is provided with a locking structure connected to its housing. The locking structure is provided with a switch member and a locking member, which cooperate with each other. The switch member can be triggered by an operator. When the switch member is not triggered, the locking member is locked, and the roller assembly is locked on the pump main body; when the switch member is triggered, the locking member is unlocked, the roller assembly is disconnected from the pump main body, and the roller assembly can be removed from the pump main body. In this way, the connection between the roller assembly and the pump main body is more convenient, since installation and removal of the roller assembly on the pump main body does not require the use of tools. The installation and removal of the roller assembly is more efficient, and it is easy to clean and maintain the roller assembly.

In order to achieve this goal, and to make the technical solutions and advantages of the embodiments of the invention clearer, the technical solutions in the embodiments of the invention will be described in the following in conjunction with the accompanying drawings. The described embodiments are, however, only some of the possible embodiments of the invention, not all of them. Based on the following description of embodiments in this invention, a person of ordinary skill in the art will be able to realize additional embodiments without needing any creative effort and such additional embodiments will thus fall within the scope of protection provided to this invention.

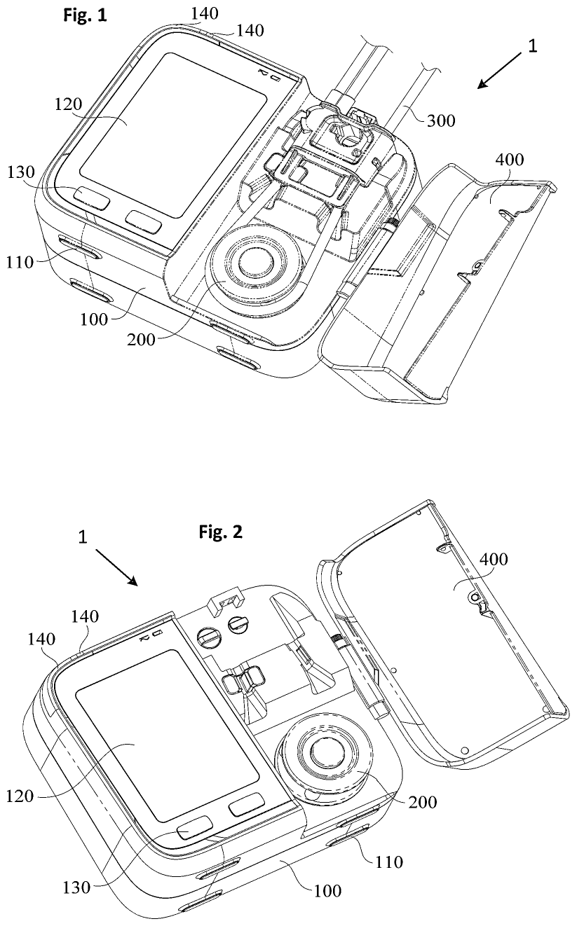

FIG. 1 shows a structural schematic diagram of an enteral feeding pump provided by an embodiment of the invention; FIG. 2 shows a structural diagram of the enteral feeding pump of FIG. 1 after removal of an infusion assembly. As shown in FIGS. 1 and 2, the enteral feeding pump 1 includes a pump main body 100, a roller assembly 200, an infusion assembly 300, and a pump door 400. The pump main body 100 is the main structure of the enteral feeding pump 1; the roller assembly 200 and the infusion assembly 300 are detachably mounted on the pump main body 100; and the pump door 400 is movably connected to the pump main body 100.

A side surface of the pump main body 100 may be an operating/work surface. An operator may face the operating surface of the pump main body 100 to operate the pump main body 100. The roller assembly 200 and the infusion assembly 300 may be mounted on the operating surface of the pump main body 100, and the pump door 400 may also cover the operating surface of the pump main body 100.

As an embodiment, the pump main body 100 may be arranged on a support base such as a tabletop. A support frame and a flexible support pad 110 may be provided on a bottom surface of the pump main body 100 to avoid hard contact between the pump main body 100 and the support base and to increase friction between the pump main body 100 and the support base, to ensure the stability of the pump main body 100, and to slow down the abrasion or scuffing of the bottom surface of the pump main body 100. One side surface of the pump main body 100 may be an operating surface, and the operating surface may, for example, be perpendicular to its bottom surface.

As shown in FIGS. 1 and 2, one side of the operating surface of the pump main body 100 may be a display control area, and the other side may be an accessory mounting area. The display control area is mainly configured to display information related to the pump main body 100, and an operator may operate in the display control area to control the operating state of the pump main body 100. The accessory mounting area is configured to install some accessories with the pump main body 100, which, together with the pump main body 100, form the enteral feeding pump 1.

A display screen 120, buttons 130, and indicator lights 140 may be provided in the display control area. The display screen 120 may occupy a large portion of the display control area, and the buttons 130 and the indicator lights 140 may be provided on the side of the display screen 120. The display screen 120 is configured to display information such as operating parameters, and function options of the enteral feeding pump 1. The buttons 130 may include buttons such as an on/off key and a return key, and the indicator lights 140 may include an operating indicator light, an alarm indicator light, and the like.

The above-mentioned roller assembly 200 and infusion assembly 300 may be mounted within the accessory mounting area, and the pump door 400 may be, for example, connected to the side wall of the pump main body 100 by a rotating shaft and located on the side where the accessory mounting area is located. The pump door 400 may cover only the accessory mounting area to protect the accessories mounted on the accessory mounting area, while the display control area is exposed beyond the pump door 400 to facilitate an operator's observation of the display screen 120 and manipulation of the pump main body 100. In an embodiment, the pump door 400 may be a transparent member, such that the operating state of the accessories within the accessory mounting area may be viewed through the pump door 400 to facilitate real-time observation of the operating condition of the enteral feeding pump 1.

As shown in FIGS. 1 and 2, the roller assembly 200 may rotate under the drive of the pump main body 100. A tube is disposed around the outer periphery of the roller assembly 200. The roller assembly 200 exerts pressure on the tube of the infusion assembly 300, so that the tube of the infusion assembly 300 is stretched. The pump main body 100 may be provided with a drive device, and the driving force of the drive device may be transmitted to the roller assembly 200, such that the drive devices causes the roller assembly 200 to rotate.

When the pump main body 100 drives the roller assembly 200 to rotate, the roller assembly 200 generates a squeezing force on the fluid within the tube of the infusion assembly 300, driving the fluid within the infusion assembly 300 to flow along the tube so as to deliver the nutrients within the infusion assembly 300 to the patient. When the roller assembly 200 stops rotating, the tube is subjected to a high pressure by the roller assembly 200. The fluid pathway within the tube is then substantially blocked, such that the fluid within the tube stops continuing to flow forward and the enteral feeding pump 1 stops delivering the nutrient to the patient.

The roller assembly 200 and the connection structure between the roller assembly 200 and the pump main body 100 are described in detail below.

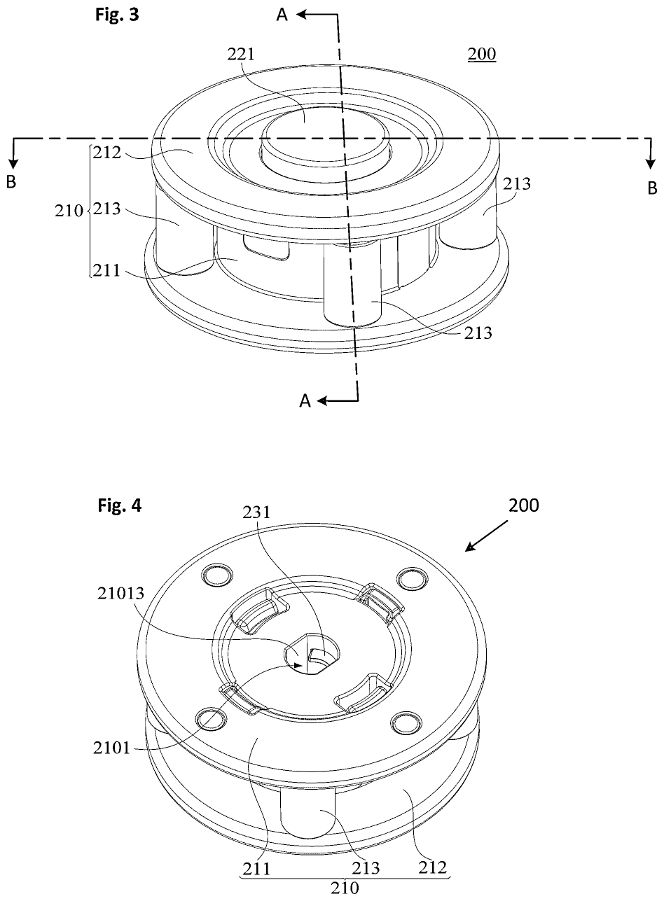

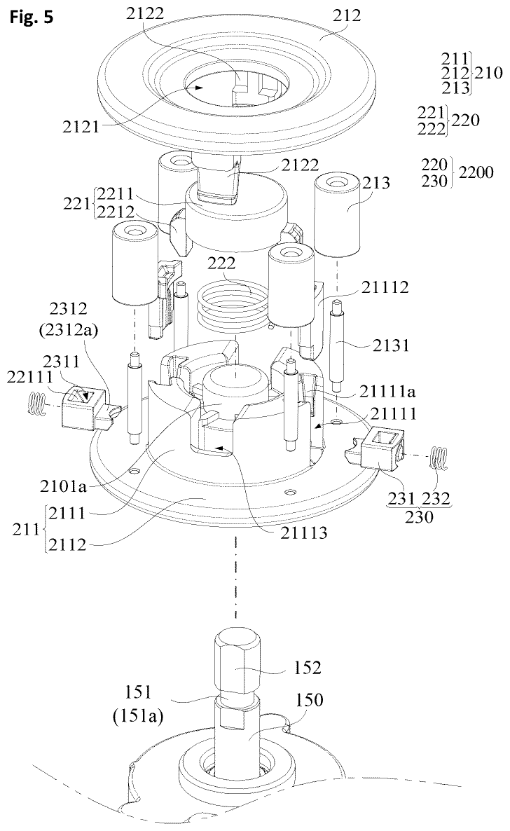

FIG. 3 shows a schematic structural diagram of the roller assembly in one view provided by an embodiment of the invention; FIG. 4 shows a different view of the schematic structural diagram of the roller assembly in FIG. 3; and FIG. 5 shows an exploded view of the roller assembly in FIG. 3.

As shown in FIGS. 3 and 4, in this embodiment, the roller assembly 200 includes a housing 210 and a locking structure 2200. The housing 210 is a main support structure of the roller assembly 200, and the roller assembly 200 is connected to the pump main body 100 by the housing 210 (comprising members 211-213). The locking structure is movably coupled to the housing 210, and the locking structure has a locked state and an unlocked state.

When the locking structure is in the locked state, the roller assembly 200 is connected to the pump main body 100, which locks the roller assembly 200 to the pump main body 100. When the locking structure is unlocked, the roller assembly 200 is disengaged from connection with the pump main body 100, which separates the roller assembly 200 from the pump main body 100. The roller assembly 200 can then be moved relative to the pump main body 100, thereby facilitating mounting the roller assembly 200 on the pump mainframe 100, or removing the roller assembly 200 from the pump mainframe 100.

As shown in FIGS. 3-5, the locking structure may comprise a switch member 221 and a locking member 231, which cooperate with each other. The switch member 221 is operable by an operator. The switch member 221 can, in particular, be triggered by the operator. The locking member 231 is configured to cooperate with the pump main body 100 to either lock or unlock the locking member 231.

When the switch member 221 is triggered, the state of the locking member 231 may be changed to switch the locking member 231 between the locked state and the unlocked state. For example, when the roller assembly 200 is assembled on the pump main body 100, in normal circumstances, the locking member 231 will be attached to the pump main body 100. In other words, when the locking member 231 is in the locked state, the roller assembly 200 is stably and reliably attached to the pump main body 100. When the switch member 221 is triggered, the switch member 221 may drive the locking member 231 separate from the pump main body 100, to enable the locking member 231 to be in the unlocked state. The roller assembly 200 may then move relative to the pump main body 100. After the switch member 221 is released from triggering, or when the switch member 221 is triggered again, the locking member 231 returns to being connected to the pump main body 100, and the locking member 231 returns to the locked state.

Compared to the prior art in which the roller is fixedly connected to the enteral feeding pump main body through screws, bolts and other fasteners, in this embodiment, the locking structure is connected to the housing 210 of the roller assembly 200 in a removable manner. The locking structure controls the connection state between the roller assembly 200 and the pump main body 100 by setting the switch member 221 and the locking member 231, which cooperate with each other. When the locking member 231 locked, the roller assembly 200 is connected to the pump main body 100. When it is necessary to separate the roller assembly 200 from the pump main body 100, the operator only needs to maneuver the switch member 221 to move the locking member 231 to the unlocked position, so that the roller assembly 200 may be detached from the pump main body 100.

With such arrangement, the roller assembly 200 is connected to the pump main body 100 in a more convenient manner, and there is no need to use a tool, specific or otherwise, when installing and removing the roller assembly 200 from the pump main body 100. The roller assembly 200 may be disassembled and assembled on the pump main body 100 in a more efficient manner, which facilitates the cleaning, maintenance, repairing, and replacing of the roller assembly 200.

In addition, the pump main body 100 may also be provided with a detecting member, which is configured to detect the state of the roller assembly 200 and to send a signal to the control unit of the pump main unit 100 when the roller assembly 200 is experiencing an abnormal condition. For example, when the roller assembly 200 is mounted on the pump main body 100, the detecting member may be provided to carry out real-time detection of the position and state of the roller assembly 200. Examples of abnormal conditions include when the rotation speed of the roller assembly 200 is abnormal, or the spacing between the roller assembly 200 and the operating surface of the pump main unit 100 is too large, or even when the roller assembly 200 is dislodged from the pump main unit 100. After receiving the signal, the control unit may autonomously control the pump main body 100 to stop the pump and send an alarm signal (e.g., a visible prompt using the alarm indicator light 140, or an audible prompt through a buzzer, or an audible and visual prompt at the same time).

As an example, the detecting member set on the pump main body 100 may be a device such as a Hall sensor, an infrared sensor, an ultrasonic sensor, a photoelectric sensor, or a microswitch. The roller assembly 200 may be provided with a magnetic member, a shading member, etc., The detecting member may thereby detect signals such as the spacing, the change of the spacing, and the change of the position of the roller assembly 200 with respect to the pump main body 100, so as to perform real-time monitoring of the in-position state of the roller assembly 200.

As shown in FIG. 5, a pump main shaft 150 extends from the operating surface of the pump main body 100. The roller assembly 200 is connected to the pump main shaft 150, and the roller assembly 200 is driven to rotate by the pump main shaft 150. It should be noted that the pump main shaft 150, which extends over the operating surface of the pump main body 100, may be connected to a drive device within the pump main body 100, which drives the pump main shaft 150 to rotate, and the pump main shaft 150 drives the roller assembly 200 to rotate.

In order for the roller assembly 200 to cooperate with the pump main shaft 150 on the pump main body 100, the housing 210 of the roller assembly 200 may be provided with a positioning groove 2101 (as shown in FIG. 4). A groove opening of the positioning groove 2101 is located on a surface of a side of the roller assembly 200 facing toward the pump main body 100, and the positioning groove 2101 extends in the rearward direction of the pump main body 100. The roller assembly 200 is sleeved outside the pump main shaft 150 by means of the positioning groove 2101; in other words, the pump main shaft 150 is inserted into the positioning groove 2101 of the roller assembly 200 to connect the roller assembly 200 to the pump main shaft 150.

As shown in FIG. 5, in order to enable the pump main shaft 150 to drive the roller assembly 200 to rotate stably, in this embodiment, an inner side wall of the positioning groove 2101 may be provided with a first positioning surface 21013 (as shown in FIG. 4). In order to cooperate with the above structure, an outer side wall of the pump main shaft 150 may be provided with a second positioning surface 152. The first positioning surface 21013 within the roller assembly 200 cooperates with the second positioning surface 152 on the pump main shaft 150, so that the roller assembly 200 is positioned on the pump main shaft 150, so as to synchronize the rotation of the roller assembly 200 with the pump main shaft 150, and to ensure stable operation of the roller assembly 200.

As an example, the first positioning surface 21013 within the positioning groove 2101 may lie in a vertical plane extending along an axial direction of the pump main shaft 150, and correspondingly, the second positioning surface 152 on the pump main shaft 150 may be a flat-cut surface. The number of the first positioning surfaces 21013 within the positioning groove 2101 may be one or two or more, and correspondingly, the number of the second positioning surfaces 152 on the pump main shaft 150 may also be one or two or more, e.g., the cross-section of the positioning groove 2101 may be a D-shape or a waist-shape, and the cross-section of the pump main shaft 150 may be a D-shape or a waist-shape.

The locking member 231 may thereby cooperate with the pump main shaft 150. When the locking member 231 is in the locked position, the locking member 231 extends into the positioning groove 2101 and is connected to the pump main shaft 150 to lock the roller assembly 200 to the pump main shaft 150, so that the roller assembly 200 is stably connected to the pump main shaft 150, and the pump main shaft 150 may drive the roller assembly 200 to rotate. When the locking member 231 is in the unlocked position, the locking member 231 may move away from the centerline (e.g., the central axis) of the positioning groove 2101 to disengage the locking member 231 from the pump main shaft 150, so that the roller assembly 200 is detached from the pump main shaft 150; this enables convenient mounting and dismounting of the roller assembly 200 on the pump main shaft 100.

As for the triggering method of the switch member 221, the switch member 221 may be triggered by pressing, toggling, or other manual operation. In other words, the switch member 221 may be a press member, a toggle member, or other structural member requiring manual operation. When the switch member 221 is not manually operated, the locking member 231 is in the locked state, and when the switch member 221 is triggered (e.g., by pressing the press member or toggling the toggle member), the switch member 221 actuates the locking member 231 to move to the unlocked state.

Of course, it is also possible to control the locking member 231 to change state in other ways. In this case, the switch member 221 may be an electronic switch, and by triggering the switch member 221 to send a control signal, the locking member 231 may be controlled to switch between the locked state and the unlocked state. In this regard, the locking member 231 may be designed as an electronically controlled locking device. For example, the locking member 231 may be automatically driven to move by an electronically controlled drive structure such as a linkage or a gear, or the locking member 231 may be driven to move relative to the pump main shaft 150 by changing the magnetism of the locking member 231.

The following are illustrations of the structure of the locking structure and its working principle, using the example of a manual operation mode in which the trigger mode of the switch member 221 is by pressing it.

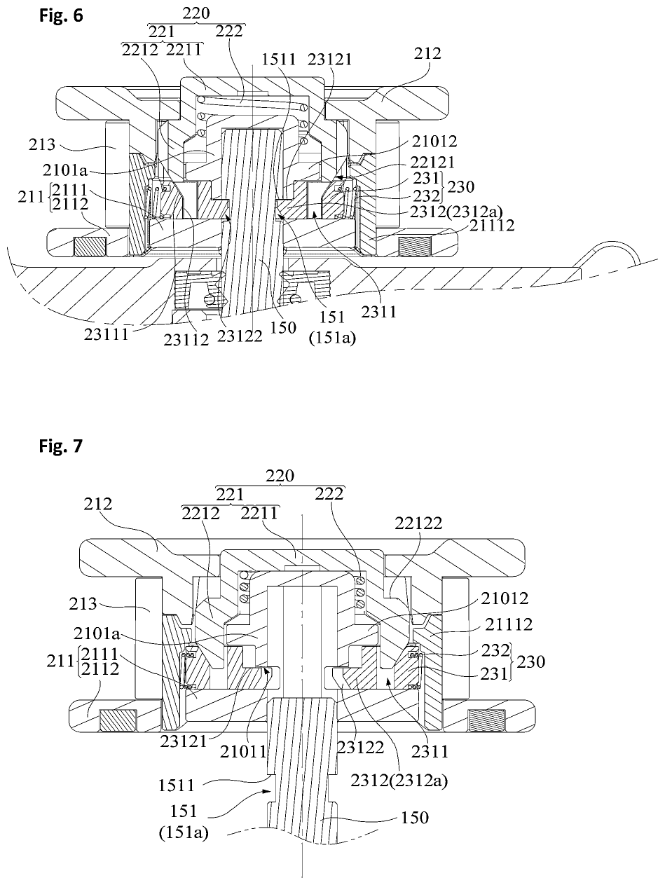

FIG. 6 shows a cross-sectional view of the roller assembly in FIG. 3 when the locking member is in the locked position; and FIG. 7 shows a cross-sectional view of the roller assembly in FIG. 3 when the locking member is in the unlocked position. As shown in FIG. 6 or FIG. 7, the housing 210 of the roller assembly 200 is provided with an accommodating cavity, in which the locking structure is installed and which can limit the locking structure by the housing 210, ensure the position of the locking structure and the accuracy of its movement, and facilitate the cooperation and connection between the locking structure and the pump main shaft 150 inserted into the roller assembly 200.

As an example, the housing 210 of the roller assembly 200 is provided with a positioning portion 2101a, which may extend from one side of the roller assembly 200 toward the other side along a direction toward the pump main body 100. An accommodating cavity is then defined between the positioning portion 2101a and the outer side wall of the housing 210. The positioning portion 2101a is located, for example, on the center axis of the roller assembly 200, and the aforementioned positioning groove 2101 may be opened on the positioning portion 2101a. In other words, the positioning portion 2101a forms a groove wall of the positioning groove 2101, and the pump main shaft 150 is inserted into the positioning groove 2101, such that the positioning portion 2101a is sleeved on the pump main shaft 150.

As shown in FIGS. 5 and 6, the housing 210 is provided with a first opening 2121, which is in communication with the accommodating cavity, and the switch member 221 is disposed through the first opening 2121. In other words, the switch member 221 extends through the first opening 2121 into the accommodating cavity. As shown in FIGS. 6 and 7, the housing 210 is further provided with a second opening 21011, which is in communication with the accommodating cavity. The second opening 21011 is in communication with the positioning groove 2101; for example, the second opening 21011 may be provided in a groove wall of the positioning groove 2101. In other words, the second opening 21011 is provided in a side wall of the positioning portion 2101a. The locking member 231 is movably disposed in the second opening 2101, and the switch member 221 is disposed in the first opening 2121. That is, the locking member 231 may extend through the second opening 21011 into the positioning groove 2101 to be connected to the pump main shaft 150, and the locking member 231 may also be movable in a direction away from the positioning groove 2101 to be detached from the pump main shaft 150.

Taking the switch member 221 as a press member as an example, in order to facilitate the operation of the switch member 221 and also facilitate the movement of the locking member 231 driven by the switch member 221, the switch member 221 may be movably connected to the top of the housing 210, and the switch member 221 may, for example, be movable in an axial direction along the housing 210. When the switch member 221 is pressed down, the switch member 221 moves in the direction toward the pump main shaft 100 and drives the locking member 231 in the direction away from the direction of the centerline (the pump main shaft 150) of the positioning groove 2101 to make the locking member 231 to the unlocked state.

In this regard, the first opening 2121 in the housing 210 may be opened at the top of the housing 210, the switch member 221 extends through the first opening 2121 into the accommodating cavity of the housing 210, and the switch member 221 is exposed in the first opening 2121 for an operator to press. As an example, the switch member 221 may protrude over the top of the housing 210 when the switch member 221 is in its “natural”, unforced state. In other words, when the switch member 221 is not depressed, the switch member 221 may protrude over the top of the housing 210, thereby reserving sufficient operating space for an operator.

The locking member 231 may be disposed within the accommodating cavity of the housing 210, and may be movable in a direction perpendicular to an axial direction of the housing 210. For example, the locking member 231 may be movable in a radial direction of the pump main shaft 150. (A slide groove may be disposed within the accommodating cavity of the housing 210 that extends in a radial direction of the pump main shaft 150, and the locking member 231 is disposed within the slide groove to guide the locking member 231 in the radial movement of the main shaft 150). The locking member 231 may move toward the pump main shaft 150 in the radial direction of the pump main shaft 150 and be connected to the pump main shaft 150 to place the locking member 231 in the locked position. The locking member 231 may also move away from the pump main shaft 150 along the radial direction of the pump main shaft 150 and be detached from the pump main shaft 150 to place the locking member 231 in the unlocked position.

In addition, in order to make the connection between the roller assembly 200 and the pump main shaft 150 smooth, at least two locking members 231 may be provided in the housing 210 of the roller assembly 200. The locking members 231 may be spaced apart around the circumference of the accommodating cavity (the circumferential direction of the pump main shaft 150), and each of the locking members 231 may be connected to a different part of the circumference of the pump main shaft 150, so as to ensure that the roller assembly 200 and the pump main shaft 150 are firmly and securely connected.

In the case that two locking members 231 are provided in the housing 210 of the roller assembly 200, the two locking members 231 may be provided symmetrically. In other words, the two locking members 231 may be located in the opposite radial direction of the pump main shaft 150. In the case that more than three locking members 231 are provided in the housing 210 of the roller assembly 200, the locking members 231 may be provided at evenly spaced intervals around the circumference of the pump main shaft 150.

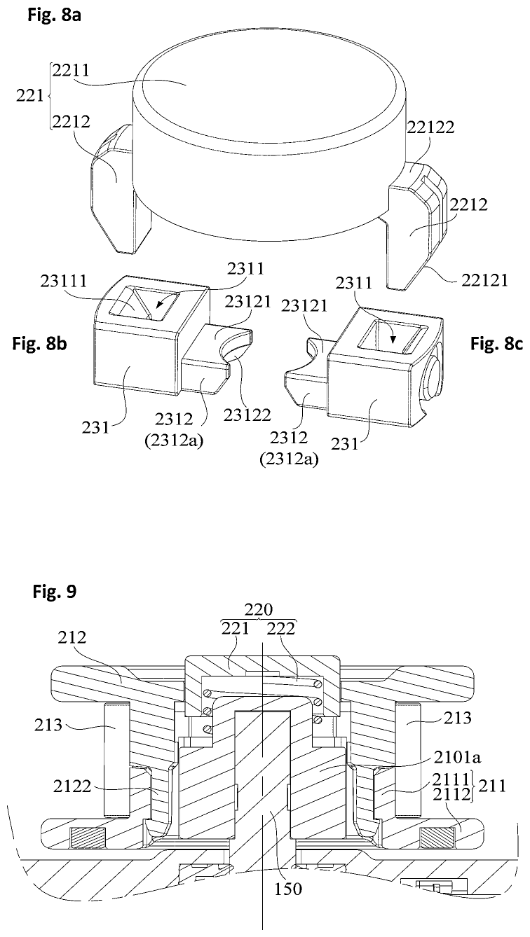

FIGS. 8a-8c together show a structural diagram of the switch member and the cooperating locking member provided by an embodiment of the invention. As shown in FIGS. 8a-8c, the switch member 221 may include a pressing portion 2211 and a driving portion 2212. The pressing portion 2211 may be a main structure of the switch member 221, and the driving portion 2212 may be connected to the pressing portion 2211, e.g., the driving portion 2212 may be integrally molded on the pressing portion 2211. As an example, the pressing portion 2211 may be a press sleeve arranged through the first opening 2121 of the housing 210 and sleeved outside of the positioning portion 2101a (as shown in FIG. 6 or FIG. 7). The driving portion 2212 may be connected to a bottom end of the pressing portion 2211, the driving portion 2212 may be cooperated one-to-one with the locking member 231, and the driving portion 2212 may abut against the locking member 231 to drive the movement of the locking member 231.

In an embodiment, the locking member 231 may be provided with a guide groove 2311, which extends from one side surface of the locking member 231 along the direction toward the switch member 221 to the other side surface. In other words, a groove opening of the guide groove 2311 may be located on the side surface of the locking member 231 toward the switch member 221. As an example, the guide groove 2311 may extend through both surfaces of the locking member 231, that is, the guide groove 2311 is a through groove opened in the locking member 231. The driving portion 2212 of the switch member 221 may be movably connected within the guide groove 2311. That is, the driving portion 2212 of the switch member 221 may be movable along the guide groove 2311 to drive the movement of the locking member 231.

The groove wall of the guide groove 2311 has an inclined first guide portion 23111. The first guide portion 23111 extends from one side surface of the locking member 231 toward the switch member 221 to the other surface, and the first guide portion 23111 is arranged at an inclined angle with respect to the axial direction of the pump main shaft 150. The driving portion 2212 of the switch member 221 is provided with a second guide portion 22121, which faces toward the first guide portion 23111. The second guide portion 22121 is matched with the first guide portion 23111, that is, the second guide portion 22121 is also arranged at an inclined angle with respect to the axial direction of the pump main shaft 150. The second guide portion 22121 may be attached to the first guide portion 23111, and is configured to slide along the first guide portion 23111 to drive the movement of the locking member 231.

As shown in FIG. 6, when the switch member 221 is not pressed, the locking member 231 is in a position closest to the pump main shaft 150, and the locking member 231 is connected to the pump main shaft 150. The locking member 231 is in the locked position, and the roller assembly 200 can be fixed to the pump main shaft 150. As shown in FIG. 7, when the switch member 221 is pressed down, the driving portion 2212 of the switch member 221 moves downwardly toward the locking member 231. The driving portion 2212 extends into the guide groove 2311 of the locking member 231, and the second guide portion 22121 of the driving portion 2212 slides along the first guide portion 23111 of the guide groove 2311 to drive the locking member 231 to move in the direction away from the pump main shaft 150, so that the locking member 231 is detached from the pump main shaft 150, and thus, the roller assembly 200 is detached from the pump main shaft 150.

As shown in FIGS. 6 and 8, as an embodiment, the first guide portion 23111 on the locking member 231 may be provided in a side groove wall of the guide groove 2311 away from the pump main shaft 150. From one end of the first guide portion 23111 toward the press member to the other end of the first guide portion 23111, the first guide portion 23111 is inclined toward the pump main shaft 150. Accordingly, the second guide portion 23111 on the switch member 221 is inclined toward the pump main shaft 150. The second guide portion 22121 on the switch member 221 may be provided on the surface of the side of the driving portion 2212 back away from the pump main shaft 150. From the end of the second guide portion 22121 toward the locking member 231 to the other end of the second guide portion 22121, the second guide portion 22121 is inclined toward a rearwards direction, away from the pump main shaft 150, and the second guide portion 22121 is attached to the first guide portion 23111.

As an embodiment, the first guide portion 23111 on the locking member 231 may be provided in a groove wall on the side of the guide groove 2311 close to the pump main shaft 150. From the end of the first guide portion 23111 toward the switch member 221 to the other end of the first guide portion 23111, the first guide portion 23111 is inclined in a direction toward the pump main shaft 150. Correspondingly, the second guide portion 22121 may be provided on a side surface of the driving portion 2212 toward the pump main shaft 150. From one end of the second guide portion 22121 toward the locking member 231 to the other end of the second guide portion 22121, the second guide portion 22121 is inclined in a direction away from the pump main shaft 150, and the second guide portion 22121 abuts against the first guide portion 23111.

Taking the first guide portion 23111 on the locking member 231 provided on a side groove wall of the guide groove 2311 away from the pump main shaft 150 as an example, as shown in FIG. 6, the side groove wall of the guide groove 2311 away from the pump main shaft 150 may also be provided with a vertical portion 23112. The vertical portion 23112 may be located on the side of the first guide portion 23111 back away from the switch member 221, and may be extended axially along the pump main shaft 150. The positioning of the vertical portion 23112 may cause the side groove wall of the guide groove 2311 to no longer be a smooth wall, and the vertical portion 23112 may limit the minimum groove width of the guide groove 2311. It may also prevent the second guide portion 22121 of the switch member 221 from continuing to slide along the locking member 231, so as to limit the range of movement of the locking member 231, and to ensure the stability and reliability of the locking member 231.

In addition, when the first guide portion 23111 of the locking member 231 is provided in the side groove wall of the guide groove 2311 away from the pump main shaft 150, the side groove wall of the guide groove 2311 close to the pump main shaft 150 may be extended along the axial direction of the pump main shaft 150, so that it is easy for the machining of the side groove wall of the guide groove 2311. In addition, the side of the guide groove 2311 close to the switch member 221 is flared, which makes it easy for the driving portion 2212 of the switch member 221 to extend into the guide groove 2311.

As shown in FIG. 6 and FIG. 7, the locking structure may further include a first elastic member 222, which may be positioned between the switch member 221 and the inner wall of the accommodating cavity. For example, the first elastic member 222 may be positioned within the switch member 221 and sleeved outside the positioning portion 2101a. One end of the first elastic member 222 abuts against the inner top wall of the switch member 221, and the other end of the first elastic member 222 abuts against the outer side wall of the positioning portion 2101a. The first elastic member 222 is configured to drive the switch member 221 to move toward the side where the first opening 2121 in the housing 210 is located.

When the switch member 221 is not pressed, the switch member 221 is located away from the locking member 231 under the elastic force of the first elastic member 222, and the locking member 231 is in the locked state (as shown FIG. 6). When the switch member 221 is pressed down, the first elastic member 222 is compressed, the switch member 221 is moved toward the locking member 231, and the driving portion 2212 of the switch member 221 moves along the guide groove 231 of the locking member 231, to drive the locking member 231 to move in a direction away from the pump main shaft 150 to transition the locking member 231 from the locked position to the unlocked position (as shown in FIG. 7). When the external force of pressing down on the switch member 221 is eliminated, driven by the elastic force generated by the compression of the first elastic member 222, the switch member 221 moves toward the first opening 2121 (the switch member 221 moves in the direction back away from the locking member 231). The driving portion 2212 of the switch member 221 gradually disengages from the guide groove 2311 of the locking member 231, and the locking member 231 may move toward the pump main shaft 150 and return to the locked state (as shown in FIG. 6).

In addition, in order to limit the switch member 221 within the accommodating cavity of the housing 210, the switch member 221 may also be provided with an abutment portion 22122, which abuts against an inner wall surface of the housing 210 when the switch member 221 is in a natural state (not triggered). For example, the abutment portion 22122 abuts against an inner wall surface at the outer periphery of the first opening 2121, so as to prevent the switch member 221 from dislodging and coming out of the housing 210. As an example, at least a portion of the driving portion 2212 may protrude on an outer wall of the pressing portion 2211, the abutment portion 22122 may be located on the top of the driving portion 2212 and the abutment portion 22122 is located on the outer periphery of the pressing portion 2211, and the pressing portion 2211 may protrude out of the first opening 2121 while the abutment portion 22122 abuts against the inner wall on the outer periphery of the first opening 2121.

Similar to the first elastic member 222 between the switch member 221 and the positioning portion 2101a, the locking structure may further include a second elastic member 232, which may be located between the locking member 231 and the inner wall of the accommodating cavity. For example, one end of the second elastic member 232 abuts against the side of the locking member 231 toward the inner side wall of the housing 210, and the other end of the second elastic member 232 abuts against the inner side wall of the housing 210 (the inner wall of the accommodating cavity). The second elastic member 232 is configured to drive the locking member 231 to move toward the side where the second opening 21011 (on the positioning portion 2101a) is located. In other words, the second elastic member 232 is configured to drive the locking member 231 toward the pump main shaft 150.

When the switch member 221 is not pressed, the locking member 231 is in a position connected to the pump main shaft 150 under the elastic force of the second elastic member 232, and the locking member 231 is in the locked state (as shown in FIG. 6). When the switch member 221 is pressed down, the switch member 221 drives the locking member 231 to move away from the pump main shaft 150, the second elastic member 232 is compressed, and the locking member 231 is moved from the locked state to the unlocked state (as shown in FIG. 7). When the external force of pressing the switch member 221 is eliminated, driven by the elastic force generated by the compression of the second elastic member 232, the locking member 231 moves in the direction toward the second opening 21011 (the locking member 231 moves in the direction toward the pump main shaft 150), and returns to the locked state (as shown in FIG. 6).

In this regard, the switch member 221 and the first elastic member 222 may together constitute a switch module 220, the locking member 231 and the second elastic member 232 may together constitute a locking module 230, and the switch module 220 and the locking module 230 together may form a locking mechanism. Both the first elastic member 222 and the second elastic member 232 may be springs, for example, both the first elastic member 222 and the second elastic member 232 are compression springs or tension springs. In other embodiments, elastic piece, elastic adhesive, and the like may be used instead of springs as the first elastic member 222 and the second elastic member 232.

As for the connection between the locking member 231 and the pump main shaft 150, as shown in FIG. 6, in this embodiment, the locking member 231 and the pump main shaft 150 may be connected by a snap-fit method. One end of the locking member 231 toward the pump main shaft 150 may be provided with a first snap-fit portion 2312, and a second snap-fit portion 151 may be provided on the pump main shaft 150. When the locking member 231 is in the locked position, the first snap-fit portion 2312 is snap-fitted with the second snap-fit portion 151, thereby realizing the connection between the locking member 231 and the pump main shaft 150.

As an embodiment, the first snap-fit portion 2312 may be a snap-fit tab 2312a protruding from an outer side wall of the side of the locking member 231 facing the pump main shaft 150, and the second snap-fit portion 151 may be a first snap-fit groove 151a provided in a side wall of the pump main shaft 150. When the locking member 231 is in the locked state, the snap-fit tab 2312a of the locking member 231 is embedded into the first snap-fit groove 151a of the pump main shaft 150, thereby realizing the snap-fit of the locking member 231 with the pump main shaft 150, and locking the roller assembly 200 to the pump main shaft 150.

The side of the snap-fit tab 2312a of the locking member 231 back away from the pump main body 100 (the side of the locking member 231 facing the switch member 221) is provided with a first limiting surface 23121. Accordingly, the groove wall of the first snap-fit groove 151a of the pump main shaft 150 facing the side of the first opening 2121 (the side of the groove wall of the first snap-fit groove 151a toward the top of the pump main shaft 150) is provided with a second limiting surface 1511. When the locking member 231 is in the locked position, the first limiting surface 23121 of the snap-fit tab 2312a abuts against the second limiting surface 1511 of the first snap-fit groove 151a to restrict the snap-fit tab 2312a of the locking member 231 within the first snap-fit groove 151a of the pump main shaft 150.

FIG. 6 illustrates how the first limiting surface 23121 on the snap-fit tab 2312a of the locking member 231, and the second limiting surface 1511 within the first snap-fit groove 151a of the pump main shaft 150, are both planar surfaces perpendicular to the axial direction of the pump main shaft 150. In other examples, from the side of the snap-fit tab 2312a away from the pump main shaft 150 to the side of the snap-fit tab 2312a close to the pump main shaft 150, the first limiting surface 23121 of the snap-fit tab 2312a may be inclined toward the top of the pump main shaft 150, and the second limiting surface 1511 of the first snap-fit groove 151a matches and fits the first limiting surface 23121 of the snap-fit tab 2312a. In this way, the locking member 231 and the pump main shaft 150 are in a barbed snap-fit arrangement, which helps prevent the locking member 231 (in the rearward direction from the pump main body 100) from disengaging from the first snap-fit groove 151a of the pump main shaft 150.

For the generally cylindrical pump main shaft 150, the first snap-fit groove 151a provided in the side wall of the pump main shaft 150 may be an arcuate groove extending in the circumferential direction of the pump main shaft 150 (as shown in FIG. 5). Matching with the first snap-fit groove 151a, the end of the limiting tab toward the pump main shaft 150 may also extend along an arc (as shown in FIG. 8) so that the limiting tab may fit with the bottom of the bottom of the arcuate groove. As an example, an arcuate groove may be annularly provided along the circumference of the pump main shaft 150, in other words, the first snap-fit groove 151a is a head-to-tail annular groove, such that the locking member 231 may extend into the first snap-fit groove 151a on the pump main shaft 150 regardless of the orientation of the locking member 231 located at any point in the periphery of the pump main shaft 150.

In addition, the end of the snap-fit tab 2312a of the locking member 231 that extends into the positioning groove 2101 (the end of the snap-fit tab 2312a toward the pump main shaft 150) may also be provided with a avoidance surface 23122 (as shown in FIG. 7), and the avoidance surface 23122 may be extended from the side of the snap-fit tab 2312a facing the pump main body 100 (the side of the snap-fit tab 2312a back away from the switch member 221) to the other side of the snap-fit tab 2312a. From one end of the avoidance surface 23122 toward the pump main body 100 to the other end of the avoidance surface 23122, the avoidance surface 23122 is inclined toward the pump main shaft 150. In this way, when the pump main shaft 150 is inserted into the positioning groove 2101 of the roller assembly 200, the avoidance surface 23122 guides the snap-fit tab 2312a to avoid the pump main shaft 150, and the top end of the pump main shaft 150 can slide along the avoidance surface 23122, which makes it easier to push the locking member 231 in the direction away from the pump main shaft 150, and facilitates the connection of the pump main shaft 150 with the roller assembly 200.

Therefore, when mounting the roller assembly 200 to the pump main body 100, it is possible to unlock the locking member 231 by relying only on the pushing action of the pump main shaft 150 on the avoidance surface 23122 of the locking member 231 without pressing the switch member 221. When the roller assembly 200 is installed, the locking member 200 is secured in the first snap-fit groove 151a of the pump main shaft 150 under the action of the second elastic member 232 to switch the locking member 200 to the locked position. Alternatively, the switch member 221 may be pressed to put the locking member 231 in the unlocked position, the roller assembly 200 may be mounted to the pump main shaft 150, and then the switch member 221 may be released so that the locking member 200 is automatically switched to the locked state.

As shown in FIGS. 5-7, the housing 210 of the roller assembly 200 may include a roller body 211 and an end cap 212. The roller body 211 and the end cap 212 are detachably connected. The end cap 212 may be connected to the top of the roller body 211, and the accommodating cavity is located between the roller body 211 and the end cap 212. In other words, the roller body 211 and the end cap 212 together enclose a space to form the above-mentioned accommodating cavity. In this way, it is easy to fabricate and mold the housing 210 of the roller assembly 200, easy to form the accommodating cavity within the housing 210, and easy to install the locking structure within the accommodating cavity.

The first opening 2121 opened in the housing 210 may be located on the end cap 212, the positioning groove 2101 within the housing 210 may be formed on the roller body 211, and the opening of the positioning groove 2101 is located on the side of the roller body 211 back away from the end cap 212.

In order to facilitate installation of the locking module 230 in the housing 210, a mounting port 21111 may be provided on a side wall of the roller body 211. The mounting port 21111 is in communication with the accommodating cavity in the housing 210, the mounting port 21111 corresponds to the locking module 230, and a detachable stopper 21112 is provided at the mounting port 21111. When installing the locking module 230, the locking module 230 may be first passed through the mounting port 21111, and after positioning the locking module 230 in the accommodating cavity, the stopper 21112 is then attached at the mounting port 21111 to be assembled into the complete roller body 211.

As an example, side walls on both sides of the mounting port 21111 on the side wall of the roller body 211 may be provided with guide grooves 21111a, and both sides of the stopper 21112 are inserted into the guide grooves 21111a on both sides of the mounting port 21111 in order to insert the stoppers 21112 into the side wall of the roller body 211.

As shown in FIGS. 5 to 7, in this embodiment, the roller assembly 200 may further include a plurality of rollers 213, which may be connected between the roller body 211 and the end cap 212, and are spaced apart along the circumference of the roller body 211 and disposed around an outer periphery of the side wall of the roller body 211. Each roller 213 may be connected between the roller body 211 and the end cap 212 by means of a rotating axle 2131, and each roller 213 is free to rotate around its own axle 2131.

The tube of the infusion assembly 300 is wound around the outer side wall of each roller 213, whereby each roller in turn squeezes the tube. When the enteral feeding pump 1 is working, under the pulling action of the tube of the infusion assembly 300, the respective roller 213 may rotate around its own axle 2131. Rolling friction is then generated between the roller 213 and the tube, which may reduce the squeezing pressure of the roller assembly 200 on the infusion assembly 300, and ensure smooth flow of the fluid in the tube of the infusion assembly 300.

In this embodiment, the roller body 211 may include a positioning portion 2101a, a main body portion 2111, and a fixed portion 2112. The fixed portion 2112 has a substantially disc-like structure, and the positioning portion 2101a and the main body portion 2111 are each connected to the fixed portion 2112. The main body portion 2111 has a substantially annular structure, and the main body portion 2111 is disposed around the positioning portion 2101a. The positioning portion 2101a and the main body portion 2111 form the above-mentioned accommodating cavity, and the positioning groove 2101 passes through the fix portion 2112 and extends into the positioning portion 2101a.

Each roller 213 is connected between the end cap 212 and the fix portion 2112 of the roller body 211. The rollers 213 are spaced apart along the circumference of the fixed portion 2112. In other words, the rollers 213 are spaced apart around the circumference of the main body portion 2111, so that the respective roller 213 can be disposed circumferentially around the outer periphery of the side wall of the roller body 211, realizing that the tube of the infusion assembly 300 is wound around each roller.

FIG. 9 shows a cross-sectional view of the roller assembly of FIG. 3 along line A-A in FIG. 5. Combined with FIG. 5 and FIG. 9, as for the connection between the roller body 211 and the end cap 212, as an embodiment, the roller assembly 200 and the end cap 212 may be connected by a snap-fit method. For example, the end cap 212 may be provided with a snap fastener 2122 that extends toward the roller body 211, and the roller body 211 may be provided with a second snap-fit groove 21113. For example, the second snap-fit groove 21113 may be opened in the roller body 211. The second snap-fit groove 21113 on the roller body 211 is adapted to the snap fastener 2122 on the end cap 212, and the snap fastener 2122 may be snapped into the second snap-fit groove 21113 to realize the connection between the end cap 212 and the roller body 211.

Of course, the roller body 211 and the end cap 212 may also be connected with fasteners such as screws or bolts. Alternatively, the roller body 211 may be welded (e.g., ultrasonically welded) to the end cap 212. Alternatively, it would also be possible for the roller body 211 to be bonded to the end cap 212.

In the description of the invention, it should be understood that the terms “center”, “longitudinal”, “lateral”, “length”, “width”, “thickness”, “up”, “down”, “front”, “back”, “left”, “right”, “vertical”, “horizontal”, “top”, “bottom”, “inside”, “outside”, etc. indicate orientations or positional relationships based on those shown in the accompanying drawings, and are intended only to facilitate the description of the invention and to simplify the description, and are not intended to indicate or imply that the device or element referred to must have a particular orientation, be constructed and operated in a particular orientation, and therefore are not to be construed as a limitation of the invention.

Finally, it should be noted that the above embodiments are only used to illustrate the technical solutions of the invention, not to limit the same. Although the invention has been described in detail with reference to the foregoing embodiments, a person of ordinary skill in the art should understand that it is still possible to make modifications to the technical solutions as documented in the foregoing embodiments, or to make equivalent substitutions for some or all of the technical features thereof; and such modifications or substitutions do not take the essence of the corresponding technical solutions out of the scope of the technical solutions of the embodiments of the invention.

1. An enteral feeding pump, comprising:
a pump main body; and
a roller assembly, that includes:
at least one roller provided for promoting flow of an infused substance through an infusion tube;
a housing; and
a locking structure connected to the housing provided for locking the roller assembly to the pump main body;
wherein:
the locking structure comprises a switch member and a locking member that cooperates with the switch member;
the locking member is selectably placed in a locked and unlocked position by action of the switch member;
wherein the roller assembly is detachably connected to the pump main body.

2. The enteral feeding pump of claim 1, wherein a positioning groove is provided on a side of the housing, and the locking member is configured to extend into the positioning groove when in the locked state, and to move in a direction away from a centerline of the positioning groove when in the unlocked state.

3. The enteral feeding pump of claim 2, wherein the housing is provided with an accommodating cavity, and the locking structure is mounted in the accommodating cavity;
the housing is further provided with a first opening and a second opening which are both connected to the accommodating cavity, and the switch member is movably disposed within the first opening; the second opening is connected to the positioning groove, and the locking member is movably disposed within the second opening.

4. The enteral feeding pump of claim 3, wherein the switch member comprises a pressing portion and a driving portion which are connected to each other; the pressing portion is movably disposed within the first opening, and the driving portion abuts against the locking member to drive the locking member to move toward a side back away from the second opening.

5. The enteral feeding pump of claim 4, wherein the locking member is provided with a guide groove and the driving portion is movably connected within the guide groove;
a groove wall of the guide groove is provided with a first guide portion that is arranged at an inclined angle, the driving portion is provided with a second guide portion that is arranged at an inclined angle, and the first guide portion is cooperated with the second guide portion to drive the locking member to move toward the side back away from the second opening.

6. The enteral feeding pump of claim 3, wherein the locking structure further comprises a first elastic member, opposite ends of the first elastic member abut against the switch member and an inner wall of the accommodating cavity, respectively, and the first elastic member is configured to drive the switch member to move toward the first opening.

7. The enteral feeding pump of claim 3, wherein the locking structure further comprises a second elastic member, opposite ends of the second elastic member abut against the locking member and an inner wall of the accommodating cavity, respectively, and the second elastic member is configured to drive the locking member toward the second opening.

8. The enteral feeding pump of claim 3, wherein the housing comprises a roller body and an end cap which are detachably connected to each other, and the accommodating cavity is disposed between the roller body and the end cap;
the first opening is provided in the end cap and the positioning groove is provided on a side of the roller body back away from the end cap.

9. The enteral feeding pump of claim 8, wherein a side wall of the roller body is provided with a mounting port connected to the accommodating cavity, and the mounting port is provided with a detachable stopper.

10. The enteral feeding pump of claim 8, further comprising a plurality of rollers, wherein the roller body comprises a main body portion and a fix portion which are connected to each other; the plurality of rollers are disposed between the fix portion and the end cap and spaced circumferentially around the main body portion, and the rollers are rotatable about their own rotational axis.

11. The enteral feeding pump of claim 8, wherein the end cap is provided with a snap fastener, and the roller body is provided with a first snap-fit groove adapted to the snap fastener.

12. The enteral feeding pump of claim 2, wherein an avoidance surface is provided at an end of the locking member extending into the positioning groove.

13. The enteral feeding pump of claim 1, wherein the pump main body is provided with a pump main shaft, and the locking member of the roller assembly is connected to the pump main shaft when the locking member is in the locked position.

14. The enteral feeding pump of claim 13, wherein the pump main shaft is provided with a second snap-fit groove, and the locking member of the roller assembly extends into the snap-fit groove when in the locked position.

15. The enteral feeding pump of claim 13, wherein:
the roller assembly is provided with a positioning groove which is inserted into and mates with the pump main shaft,
an inner side wall of the positioning groove is provided with a first locating surface, an outer side wall of the pump main shaft is provided with a second locating surface, and
the first locating surface mates with the second locating surface, whereby the pump main shaft drives the roller assembly to rotate.

16. The enteral feeding pump of claim 1, wherein the pump main body is further provided with a detecting member that is configured to detect a state of the roller assembly and send a signal to a control unit of the pump main body in case of abnormality of the roller assembly.
